(19) United States
(12) Patent Application Publication
(10) Pub. No.: US 2009/0210969 A1
(43) Pub. Date: Aug. 20, 2009

(54) ANTIPORTER GENE FROM PORTERESIA COARCTATA FOR CONFERRING STRESS TOLERANCE

(75) Inventors: Ajay Parida, Chennai (IN); Kizhakedath Praseetha, Chennai (IN)

Correspondence Address: LOWE HAUPTMAN HAM & BERNER, LLP, 1700 DIAGONAL ROAD, SUITE 300, ALEXANDRIA, VA, 22314, US

(73) Assignee: M.S. SWAMINATHAN RESEARCH FOUNDATION, Chennai (TW)

(21) Appl. No.: 11/997,718

(22) PCT Filed: Jul. 31, 2006
(86) PCT No.: PCT/IN06/00271

§ 371 Date: Aug. 14, 2008

(30) Foreign Application Priority Data
Aug. 3, 2005 (IN) 1052/CHE/2005

(51) Int. Cl. C12N 15/82; A01H 5/00; C12N 15/29; C12N 1/21; C12N 1/19

(52) U.S. Cl. 800/294; 800/278; 800/298; 800/317.3; 800/317.4; 800/312; 800/306; 800/320.2; 800/320.1; 800/320.3; 800/320; 536/23.1; 536/23.6; 435/320.1; 435/252.3; 435/252.33; 435/254.2

(57) ABSTRACT

The present invention relates to isolation and characterization of a cDNA corresponding to Na⁺/H⁺ antiporter gene from Porteresia coarctata, the deduced protein of the said gene and its promoter region capable of conferring tolerance to abiotic stress in plants. The present invention also relates to cloning the complete cDNA sequence corresponding to Na⁺/H⁺ antiporter gene from Porteresia coarctata. The present invention also relates to isolating the promoter for Na⁺/H⁺ antiporter gene from Porteresia coarctata. The invention further provides a method for producing abiotic stress tolerant transgenic plants. Further, the invention relates to salt tolerant transformed plants of rice over-expressing the Na⁺/H⁺ antiporter gene from Porteresia coarctata.

### FIELD OF INVENTION

This invention relates to plant genes useful for the genetic manipulation of plant characteristics. More specifically, the invention relates to isolation and characterization of cDNA corresponding to antiporter gene from Porteresia coarctata that confers abiotic stress tolerance to plants, a method of producing abiotic stress-tolerant transgenic plants by expression of the said Porteresia coarctata antiporter gene, and a transformed plant containing Porteresia coarctata antiporter gene.

### BACKGROUND OF THE INVENTION

In salt tolerant plants, sodium extrusion from the cytosol and compartmentalization in vacuoles are key processes for Na⁺ detoxification and cellular osmotic adjustment. These plants accumulate Na⁺ in vacuoles through the activity of a tonoplast transmebrane protein called Na⁺/H⁺ antiporter. The first gene for a plant tonoplast Na⁺/H⁺ antiporter, AtNHX1, was isolated from Arabidopsis and shown to increase plant tolerance to NaCl.

The central importance of vacuolar sequestration has recently been underlined by experiments in which constitutive over-expression of vacuolar transporters has greatly increased salinity tolerance of a range of species. Over expression of an Arabidopsis vacuolar Na⁺/H⁺ antiporter (NHX1) increased salinity tolerance of Arabidopsis, tomato and Brassica napus. Contrary to the conception that multiple traits introduced into crop plants by breeding is the only way to obtain salt tolerant plants, the present invention showed that the modification of a single trait significantly improved the salinity tolerance of these plants. These results demonstrate that with a combination of breeding and transgenic approach it could be possible to produce salt tolerant crops with far fewer target traits than anticipated.

Porteresia coarctata(Roxb) Tateoka is a halophytic wild relative of rice native to coastal saline areas of Bangladesh, India and Pakistan and parts of the Indo-China peninsula. P. coarctata has mechanisms for tolerating salt concentrations that could kill even the most salt-tolerant rice cultivar within 2 d (>150 mM) and is thus a good source for mining gene(s) for salt and submergence tolerance.

### SUMMARY OF INVENTION

The present invention relates to isolation and characterization of a cDNA corresponding to Na⁺/H⁺ antiporter gene from Porteresia coarctata, the deduced protein of said gene capable of conferring tolerance to abiotic stress to plants. The invention also provides a method for producing abiotic stress tolerant transgenic plants. Further, the invention relates to salt tolerant transformed crop plants over-expressing the Na⁺/H⁺ antiporter gene from Porteresia coarctata. The invention also describes a drought stress tolerant transformed crop plants over-expressing the Na⁺/H⁺ antiporter gene from Porteresia coarctata.

The present invention also relates to isolation and functional characterization of promoter of Na⁺/H⁺ antiporter gene from Porteresia coarctata.

One embodiment of the present invention relates to an isolated DNA fragment coding for antiporter gene comprising a polynucleotide sequence shown in SEQ ID NO: 1, wherein expression of said polynucleotide sequence in plant results in conferring tolerance to abiotic stress as compared to a corresponding wild type plant.

Another embodiment of the present invention discloses a polypeptide comprising an amino acid sequence as shown in SEQ ID NO: 2, said polypeptide is encoded by a DNA fragment of antiporter gene comprising a polynucleotide sequence as shown in SEQ ID NO: 1. Further the over expression of antiporter gene confers tolerance to abiotic stress.

One aspect of the invention pertains to a recombinant vector comprising of a regulatory sequence operably linked to the polynucleotide sequence set forth in SEQ ID NO: 1.

Another aspect of the invention is directed to a method for conferring tolerance to abiotic stress in a plant comprising transforming the plant with a recombinant vector to produce transformed plant cells, culturing the transformed plant cells to obtain an abiotic stress tolerant plant. Further the abiotic stress factor is either salt stress or drought stress.

In a preferred embodiment, the present invention teaches a method of producing a transgenic plant by Agrobacterium mediated transformation method comprising steps of:
a. obtaining suitable explants from said plant,
b. co-cultivating the explants with an Agrobacterium strain that comprises of a recombinant vector comprising an antiporter gene having sequence as shown in SEQ ID NO: 1 to produce transformed plant cells,
c. culturing the transformed plant cells to produce the abiotic stress tolerant plant.

One embodiment of the present invention is directed to a transgenic plant comprising polynucleotide sequence as shown in SEQ ID NO: 1.

One aspect of the present invention provides a monocotyledonous or dicotyledonous transgenic plant comprising of a polynucleotide sequence as set forth in SEQ ID NO: 1 in sense direction.

One aspect of the present invention provides a monocotyledonous or dicotyledonous transgenic plant comprising of a polynucleotide sequence as set forth in SEQ ID NO: 1 in anti-sense direction.

In one aspect, the present invention pertains to an isolated promoter functional in plant cells comprising a polynucleotide sequence set forth in SEQ ID NO: 3.

In another aspect, the present invention describes an isolated promoter functional in plant cells comprising a polynucleotide sequence having at least 200 contiguous nucleotides of the polynucleotide sequence as shown in SEQ ID NO: 3.

The invention also pertains to a recombinant vector comprising a promoter having polynucleotide sequence as shown in SEQ ID NO: 3 operably linked to a heterologous DNA sequence of interest.

One aspect of the present invention discloses a method of producing a transgenic plant comprising transforming the plant with a recombinant vector operably linked to a heterologous DNA sequence of interest, wherein said recombinant vector comprises of a promoter having polynucleotide sequence as shown in SEQ ID NO: 3.

Further the invention also relates to a transgenic plant comprising a promoter having polynucleotide sequence as shown in SEQ ID NO: 3.

### BRIEF DESCRIPTION OF ACCOMPANYING DRAWINGS

FIG. 1: pCambia1301 T-DNA region containing PcNHX in sense direction under CaMV 35 S

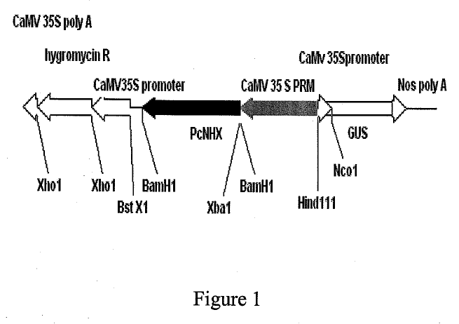

FIG. 2: pCambia1301 T-DNA region containing PcNHX in anti-sense direction under CaMV 35 S

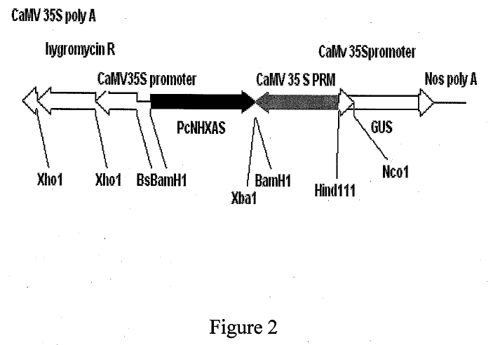

FIG. 3: pCambia1301 T-DNA region containing PcNHX promoter fused to GUS gene

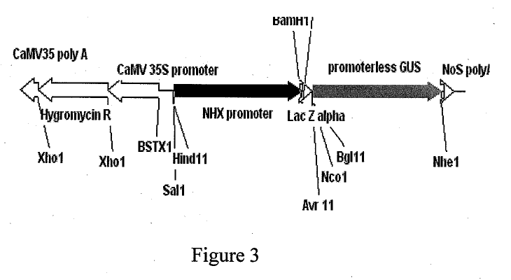

### DETAILED DESCRIPTION OF THE PREFERRED EMBODIMENTS

The invention relates to isolation and characterization of a cDNA fragment corresponding to Na⁺/H⁺ antiporter gene (PcNHX1) from Porteresia coarctata, its promoter region and the deduced protein of said gene capable of conferring tolerance to abiotic stress in a plant. It also relates to a method of subjecting seedlings of Porteresia coarctata to acute salt stress under conditions critical for expression of the transcripts of Na⁺/H⁺ antiporter gene. The invention also relates to a method of conferring tolerance to abiotic stress in plant by Agrobacterium-mediated transformation. Further, the invention relates to salt tolerant transformed crop plants over-expressing the Na⁺/H⁺ antiporter gene from Porteresia coarctata.

The present invention also pertains to isolation and functional characterization of promoter region of Na⁺/H⁺ antiporter gene (PcNHX1) from Porteresia coarctata capable of initiating transcription of a DNA sequence to which it is operably linked. The isolated promoter sequences can be used to create recombinant DNA molecules for selectively modulating expression of any operatively linked gene. These isolated promoter sequences have the biological activity of expressing operably linked nucleic acid molecules when the plant is exposed to a stressful environment.

One embodiment of the present invention relates to an isolated DNA fragment coding for antiporter gene (PcNHX1) comprising a polynucleotide sequence set forth in SEQ ID NO: 1, wherein expression of said polynucleotide sequence in plant results in conferring tolerance to abiotic stress as compared to a corresponding wild type plant. Further the abiotic stress is either salt stress or drought stress.

Another embodiment of the present invention discloses a polypeptide comprising an amino acid sequence set forth in SEQ ID NO: 2, said polypeptide is encoded by a DNA fragment of antiporter gene comprising a polynucleotide sequence as shown in SEQ ID NO: 1. Further the expression of said gene confers tolerance to abiotic stress.

One aspect of the invention pertains to a recombinant vector comprising of a regulatory sequence operably linked to the polynucleotide sequence as shown in SEQ ID NO: 1.

Another aspect of the invention pertains to a recombinant vector comprising of a regulatory sequence operably linked to at least 250 contiguous nucleotides of the polynucleotide sequence as shown in SEQ ID NO: 1.

Another aspect of the invention provides the recombinant vector is a recombinant plant transformation vector.

In another embodiment, the present invention provides method for construction of recombinant plant transformation vector namely PcNHX-1301, PcNHX AS-1301, PcNHX-PRM-1301, and PcPRM-1391z. Further the plant transformation vector is either pCAMBIA 1301 or pCAMBIA 1391Z.

The invention also pertains to a recombinant vector comprising a regulatory sequence selected from a group consisting of CaMV 35S promoter, actin promoter, maize ubiquitin promoter, and alcohol dehydrogenase promoter.

Another embodiment of the invention describes host cell comprising the recombinant vector. Further, the host cell is selected from a group consisting of E. coli, Agrobacterium and yeast.

Another embodiment of the present invention employs a host cell selected from the group of E. coli strains such as JM101, DH5α, BL21, HB101, and XL1-Blue to produce recombinant host cells.

Another embodiment of the present invention employs a host cell selected from the group of Agrobacterium strains such as LBA4404, EHA101, EHA105, GV3101 and A 281 to produce recombinant host cells.

One embodiment of the invention is directed to a method for conferring tolerance to abiotic stress in a plant comprising transforming the plant with a recombinant vector to produce transformed plant cells, wherein the recombinant vector comprises a polynucleotide sequence as shown in SEQ ID NO: 1, culturing the transformed plant cells to obtain an abiotic stress tolerant plant. Further, the abiotic stress factor is either salt stress or drought stress.

Yet another embodiment of the invention discloses a method of transformation employed to transform plants which is selected from a group consisting of Agrobacterium mediated transformation, particle bombardment, vacuum-infiltration, in planta transformation.

In yet another embodiment, the present invention provides a method of producing a transgenic plant by Agrobacterium mediated transformation method comprising steps of:
a) obtaining suitable explants from a plant,
b) co-cultivating the explants with an Agrobacterium strain that comprises of a recombinant vector comprising a polynucleotide sequence as shown in SEQ ID NO: 1 to produce transformed plant cells,
c) culturing the transformed plant cells to produce the abiotic stress tolerant plant.

One aspect of the present invention pertains to a plant used for transformation by a recombinant vector comprising a polynucleotide sequence as shown in SEQ ID NO 1. Further the plant used for transformation includes, but not limited to, monocots and dicots. More preferably the dicot plant is selected from a group consisting of tobacco, tomato, pea, soybean, Brassica, chickpea, pigeon pea. The monocot plant is selected from a group consisting of rice, maize, wheat, barley and sorghum. Protocols and procedures for plant transformation may vary depending upon the plant species and type of plant tissues.

Still another aspect of the invention relates to the explant used for transformation is selected from a group consisting of cotyledons, hypocotyls, leaves, stem and roots.

One embodiment of the present invention provides a transgenic plant having tolerance to abiotic stress comprising polynucleotide sequence as shown in SEQ ID NO: 1 in sense direction. It also relates to the progeny of said transgenic plant.

Yet another embodiment of the present invention describes a transgenic plant having tolerance to abiotic stress comprising polynucleotide sequence as shown in SEQ ID NO: 1 in anti-sense direction. Further it also relates to progeny of said transgenic plant.

Further the transgenic plant includes, but not limited to, dicots or monocots. More preferably the dicot plant is selected from a group consisting of tobacco, tomato, pea, soybean, Brassica, chickpea, pigeon pea. The monocot plant is selected from a group consisting of rice, maize, wheat, barley and sorghum.

One preferred embodiment of the present invention pertains to an isolated promoter functional in plant cells comprising a polynucleotide sequence as shown in SEQ ID NO: 3.

Yet another embodiment of the present invention provides a polynucleotide sequence having at least 200 contiguous nucleotides of the polynucleotide sequence as set forth in SEQ ID NO: 3.

The invention also pertains to a recombinant vector comprising said promoter operably linked to a heterologous DNA sequence of interest.

The invention further describes the heterologous DNA sequence encodes a protein selected from a group consisting of insect resistance protein, a bacterial disease resistance protein, a fungal disease resistance protein, a viral disease resistance protein, a nematode disease resistance protein, a herbicide resistance protein, a protein affecting grain composition or quality, a selectable marker protein, a screenable marker protein, a protein affecting plant agronomic characteristics, and a stress resistance protein.

One preferred embodiment of the present invention is directed to a transgenic plant comprising a plant functional promoter having a polynucleotide sequence as shown in SEQ ID NO: 3. It also pertains to progeny of said transgenic plant.

In another preferred embodiment, the invention teaches a transgenic plant comprising a plant functional promoter comprising a polynucleotide sequence having at least 200 contiguous nucleotides of the polynucleotide sequence as shown in SEQ ID NO: 3. Further it also provides the progeny of said transgenic plant.

One aspect of the present invention discloses a method of producing a transgenic plant comprising transforming the plant with a recombinant vector wherein, said recombinant vector comprises a promoter sequence as shown in SEQ ID NO: 3 and is operably linked to a heterologous DNA sequence of interest.

Yet another aspect of the present invention provides method of producing a transgenic plant comprising promoter region having sequence as shown in SEQ ID NO: 3 wherein, the transformation method is selected from a group consisting of Agrobacterium mediated transformation, particle bombardment, vacuum-infiltration, in planta transformation.

Still another aspect of the present invention describes Agrobacterium mediated transformation method to transform plant with a recombinant vector comprising promoter as shown in SEQ ID NO: 3.

Yet another aspect of the present invention teaches a method of producing a transgenic plant by Agrobacterium mediated transformation method comprising steps of:
a) obtaining suitable explants from said plant,
b) co-cultivating the explants with an Agrobacterium strain that comprises of a recombinant vector comprising a promoter as shown in SEQ ID NO: 3 to produce transformed plant cells,
c) culturing the transformed plant cells to produce the abiotic stress tolerant plant.

Further aspect of the present invention pertains to a plant used for transformation by a recombinant vector comprising a promoter having a polynucleotide sequence as shown in SEQ ID NO: 3. Further the plant used for transformation includes, but not limited to, monocots and dicots. More preferably the dicot plant is selected from a group consisting of tobacco, tomato, pea, soybean, Brassica, chickpea, pigeon pea. The monocot plant is selected from a group consisting of rice, maize, wheat, barley and sorghum.

### 1) Plant Growth Conditions

A preferred embodiment of the present invention relates to a method for growing Porteresia coarctata seedling under conditions of salt stress and ABA stress. EXAMPLE 1 gives a detailed account of conditions in which the Porteresia coarctata plants were grown.

### 2) RNA Isolation

Another embodiment of the invention relates to isolation of total RNA from P. coarctata seedlings or leaf material by using the method as provided by Chomczynski and Sacchi (1987). Other methods well known in the art can also be used for total RNA isolation. The RNA isolation from P. coarctata seedling can also be carried out using commercially available plant RNA isolation kits. poly (A+) mRNA was isolated by affinity chromatography on oligo (dT)-cellulose as described by Sambrook et al. (1989). EXAMPLE 2 provides the detailed procedure.

2) Construction of cDNA Library of Porteresia coarctata

Yet another embodiment of the present invention relates to a directionally cloned cDNA library. This library was constructed in Sal I (5( )/Not I (3( ) sites of pSPORT vector (Invitrogen) using poly (A+) RNA as described above. The detailed methodology on construction of cDNA library is discussed in EXAMPLE 3. Several commercially available kits are available for cDNA synthesis from (A+) enriched RNA well known to the people skilled in the art. Kits for cloning cDNA inserts both directionally and randomly are also well known and can be employed.

3) Screening of cDNA Library of Porteresia coarctata

Still another embodiment of the present invention relates to isolating the clone for antiporter gene from the cDNA library of Porteresia coarctata by employing a heterologous probe from NHX1 gene from Arabidopsis thaliana through stepwise screening methodology well known to the people skilled in art. Plasmid DNA from the colonies showing intense signal was isolated and purified by PEG precipitation method.

The methodological details pertaining to screening of cDNA library is provided in EXAMPLE 4.

### 4) Sequence Characterization and Phylogenetic Analysis of PcNHXI

Still another embodiment of the present invention relates to deriving a complete nucleic acid sequence of both strands of the full length cDNA of PcNHX1. This was determined using the dideoxy chain termination method (Sanger et al., 1977) with an ABI 310 automated DNA sequencer (Perkin-Elmer) and M13 and pUC18 forward and reverse primers. The nucleic acid sequence for PcNHX1 is shown in SEQ ID NO: 1.

This sequence was used for conducting homology searches against the sequences deposited in databases annotated and curated at NCBI (National Centre for Biotechnology Information) and its mirror sites. Nucleotide and amino acid alignments were performed using BLAST. Nucleic acid alignments were performed using BLASTN algorithm (Basic Local Alignment Search Tool for nucleotide; Altschul et al., 1997), wherein nucleotide query sequence is searched against nucleotide database.

Protein translations from nucleotide query sequence followed by amino acid sequence alignments for deduced protein against protein database was performed using BLASTX. The search result indicates that the gene codes for an antiporter protein.

5) Random Amplification of cDNA Ends

Yet another embodiment of the present invention relates to getting the sequence information at the 5′ end of the partial PcNHX1 clone. 5′RACE (Rapid Amplification of cDNA ends) was performed according to the SMART™ RACE kit from Clontech. These methods for procuring 5′ or 3′ sequence information are well known in the art. Detailed procedure followed is given in EXAMPLE 5.

### 6) Isolation of the PcNHX1 Promoter Region by TAIL-PCR

Yet another embodiment of the present invention relates to isolating the promoter sequences corresponding to PcNHX1 gene using TAIL PCR which is an efficient way of identifying flanking genomic regions. This method is well known to the people skilled in the art. The flanking regions can also be isolated by screening genomic libraries using cDNA as a probe, which is a time consuming process. The DNA sequence of the promoter is shown as SEQ ID NO: 3. The detail of the methodology followed is given in EXAMPLE 6.

### 7) Primer Extension for Mapping Transcription Initiation Site

A preferred embodiment of the present invention relates to extending the primers for mapping the 5′-end of transcripts for determination of the precise start site(s) for transcription. The detail of the methodology followed is given in EXAMPLE 7. The analysis revealed a transcription start site 234 bp upstream of the translation start site.

### 8) Kinetics of Induction of PcNHX1 in Response to Abiotic Stresses

Yet another embodiment of the present invention relates to studying the effect of abiotic stress on the expression of the PcNHX1 in Porteresia coarctata by methods well known to the people skilled in art. Further said abiotic stress comprises of salt stress and ABA stress (see EXAMPLE 8).

### 9) Construction of Plant Transformation Vectors

Yet another embodiment of the present invention relates to cloning the PcNHX1 insert in the sense direction into the BamH1 site of the binary vector pCAMBIA 1301 downstream of CaMV 35S promoter. The gene was also cloned in the antisense orientation into the same restriction site in the same vector. A third construct was made by cloning the PcNHX1 promoter sequence upstream of the PcNHX gene in pCAMBIA 1301. The PcNHX promoter was cloned in the Sal1 and BamH1 sites of the promoter fusion vector pCAMBIA 1391Z upstream of a promoter less GUS gene. See EXAMPLE 9 for detail procedure.

### 10) Transformation of Tobacco Using Agrobacterium

Yet another embodiment of the present invention relates to transformation of tobacco explants by standard Agrobacterium method which is well known to the people skilled in art. The transformed tobacco plant confers tolerance to abiotic stress. The details on Agrobacterium co-cultivation are provided in EXAMPLE 10.

### 11) Transformation of Rice Using Agrobacterium

One embodiment of the invention relates to the production of the transformed rice carrying antiporter gene from P. coarctata to confer the salt stress tolerance in rice plant. Transformation of rice calli was performed by standard Agrobacterium method which is well known to the people skilled in art. Detailed procedure of rice transformation is given in EXAMPLE 11.

### 12) Transformation of Rice Through Particle Bombardment

Yet another embodiment of the present invention relates to transforming two cultivated varieties of rice, Pusa Basmati and IR-20 through particle bombardment by employing a standard method for transformation of rice well known to the people skilled in the art. Details of the method are provided in EXAMPLE 12.

### 13) Screening of the Transformed Plants

Yet another embodiment of the present invention relates to screening of the rice and tobacco transgenic plants harboring the construct by employing methods known in the art.

### 14) Analyzing Salt Tolerance in Tobacco and Rice Transgenics

Yet another embodiment of the present invention is directed to analyzing the salt tolerance conferred by over-expressing PcNHX1 in tobacco transgenic by performing whole plant salt stress treatments (See EXAMPLE 13).

Yet another embodiment of the present invention is directed to analyzing the salt tolerance conferred by over-expressing PcNHX1 in rice transgenics by performing whole plant salt stress treatments (See EXAMPLE 13).

### 15) GUS Assay for PcNHX1 Promoter Activity in Tobacco

Yet another embodiment of the present invention relates to checking the functionality of PcNHX1 promoter in transgenic plants. Detailed procedure is mentioned in EXAMPLE 14.

### EXAMPLES

The examples given are merely illustrative of the uses, processes and products claimed in this invention, and the practice of the invention itself is not restricted to or by the examples described.

### Example 1

### Plant Growth Parameters

### Plant Growth Conditions

For constructing cDNA library, RT-PCR and RACEs, Porteresia coarctata seedlings were grown for one month and treated with 0.5M NaCl for 48 h.

For studying induction kinetics of PcNHX1 through Northern analysis in response to salt and ABA stress, the P. coarctata seedlings were acclimatized for 72 hours in 0.5× MS nutrient solution and then exposed to salt stress and ABA treatment. Leaf tissue was frozen for RNA isolation.

Salt stress: Plants were stressed with 0.5× MS containing 0.5M NaCl and leaf tissue frozen at 6 h, 12 h, 24 h, 48 h NaCl treatment and 12 h and 24 h after salt withdrawal.

ABA treatment: Plants were stressed with 0.5× MS containing 100 M ABA and leaf tissue frozen at 6 h, 12 h, 24 h, 48 h NaCl treatment and 12 h and 24 h after ABA withdrawal. The method being critical for specifically up-regulating the expression levels of transcripts of antiporter gene (PcNHX1) gene, thereby allowing efficient cloning of antiporter (PcNHX1) gene as a cDNA fragment.

### Example 2

### RNA Isolation

Total RNA was isolated from pooled leaf tissue of Porteresia coarctata seedlings according to Chomczynski and Sacchi (1987). Leaf tissue was harvested from pooled plants and five grams of tissue was macerated in liquid nitrogen and suspended in 18 ml of RNA extraction buffer. To the slurry, 1.8 ml of 2 M sodium acetate (pH 4.0), 18 ml of water saturated phenol and 3.6 ml of 49:1 chloroform:isoamyl alcohol were sequentially added and mixed by inversion. The contents were mixed and cooled on ice for 15 minutes. Finally, the suspension was centrifuged at 10,000×g for 10 minutes at 4° C. After centrifugation, the aqueous phase was transferred to a fresh tube and mixed with equal volume of ice-cold isopropanol and incubated at −20° C. for 1 hour. The samples were centrifuged at 10,000×g for 20 minutes at 4° C. and the pellet was dissolved in 5 ml of RNA extraction buffer. The RNA was again re-precipitated with equal volume of ice-cold isopropanol. The pellet was washed in 70% ethanol and finally dissolved in DEPC water. Purity of the RNA preparation was checked spectrophotometrically by measuring A260/A280 ratio. An A260/A280 value between 1.8 and 2.0 suggested that the RNA was intact and pure. Finally, the total RNA in the samples was estimated by measuring A260. Poly (A+) mRNA was isolated by affinity chromatography on oligo (dT)-cellulose as described by Sambrook et al. (1989).

### Example 3

### Construction of cDNA Library

The directionally cloned cDNA library was constructed in Sal I (5( )/Not I (3( ) sites of pSPORT vector (Invitrogen) using poly (A+) RNA as described in EXAMPLE 2. For first strand synthesis, 5 μg of mRNA and 1 μg of primer-adapter as shown in SEQ ID NO 4 was taken in 8 μl volume in a RNAase free eppendorf tube, incubated at 70° C. for 10 minutes, and quickly chilled on ice. After brief centrifugation, 4 μl of 5× first strand buffer (250 mM Tris-HCl (pH-8.3), 175 mM KCl and 15 mm MgCl2), 2 μl of 0.1 M DTT and 1 μl of 10 mM dNTP mix was added. After incubation at 37° C. for 2 min, 5 μl of reverse transcriptase enzyme (200 U/μl) Superscript II RT, Life technologies, USA) was added and incubated at 37° C. for 1 hr. This was followed by second strand synthesis. In this step, the 20 μl first strand reaction mixture, 91 μl of water, 30 μl of 5× second strand buffer (100 mm Tris-HCl, pH 6.9; 450 mM KCl, 23 mM MgCl2; 0.75 b-NAD+; 50 mM (NH4) 2SO4, 3 μl of 10 mM dNTP mix, 1 μl of E. coli DNA ligase (10 u/μl, Gibco-BRL), 4 μl of E. coli DNA polymerase (10 u/μl, Gibco-BRL) and 1 μl of E. coli RNAase H (2 u/μl, Gibco-BRL) were added and incubated at 16° C. for 2 hrs. Then, 2 μl of T4 DNA polymerase (5 U/μl) was added the incubation was continued at 16° C. for 5 min. The reaction was terminated by adding 10 μl of 0.5 M EDTA. The double strand cDNAs were purified by phenol; chloroform extraction and precipitated using 0.5 volume of 7.5 M ammonium acetate and 2.5 volume of ethanol. The pellet was washed with 70% ethanol; air dried and re-suspended in 25 μl DEPC treated water. This was followed by Sal1 adapter ligation. The cDNA in 25 μl water was placed on ice and 10 μl of 5× T4 DNA ligase buffer, 10 Sal1 adapters and 5 μl of T4 DNA ligase (1 U/μl, Gibco-BRL) were added. The reaction mixture was gently mixed and incubated at 16° C. for 16 hr. The sample was purified by phenol: chloroform extraction and precipitated. The pellet was given a 70% ethanol wash and re-suspended in 41 μl water and digested with Not1 to generate the cDNAs for Sal1 and Not1 cohesive end at 5′ and 3′ end, respectively. The next step involved size fractionation of cDNA. The cDNA after Not1 digestion was purified and re-suspended in 120 μl 1× ligation buffer and size fractionated on the spun column, Sepharose CL-4B (Amersham-Pharmacia Biotech). 2 μl from this was taken for each ligation reaction. 50-μg pSPORT 1 plasmid vector containing cohesive end for the restriction enzymes Sal 1 and Not1 was ligated to 2 μl of size fractionated cDNA.

(Primer)

SEQ ID NO 4

5′-GACTAGT TCTAGA TCGCGA GCGGCCGC CC (T) 15-3′

The ligated cDNA library was transformed into E. coli DH5 alpha. A library of approximately 105 recombinants was obtained. Approximately 2000 individual colonies were pooled and fifty such pools contained (P1-P50) about 1×105 clones. Several commercially available kits are available for cDNA synthesis from (A+) enriched RNA well known to the people skilled in the art. Kits for cloning cDNA inserts both directionally and randomly are also well known and can be employed.

### Example 4

### Screening of P. coarctata Leaf cDNA Library

A heterologous probe using NHX1 gene from Arabidopsis thaliana was for isolating the clone for antiporter gene from the cDNA library of Porteresia coarctata. The NHX1 gene was derived from Arabidopsis thaliana cDNA through RT-PCR. The primers for RT-PCR reaction were designed after aligning yeast and plant Na+/H+ amino acid antiporter sequences and identifying highly conserved blocks. RT-PCR generated a 1.5 Kb fragment which was cloned into blunt end cloning vector. The sequence of the cloned RT-PCR product corresponding to AtNHX1 was confirmed by sequencing and was subsequently used as a probe. Total RNA was isolated from Arabidopsis seedlings by a protocol described by Chomczynski and Sacchi (1987).

For screening for the plasmid carrying the antiporter cDNA from Porteresia coarctata, plasmid DNA from 50 cDNA pools (P1-P50) was dot blotted on a nitrocellulose membrane and this was used as a master blot for the primary screening. The master blot was hybridized with the AtNHX1 probe using standard methods well known in the art and hybridization washes were performed at high stringency. Two pools (P27, P37) in the master blot showed intense hybridization signals. The plasmid DNA from pools P27 and P37 were transformed to E. coli DH5. The transformants were serially diluted and plated. The plates containing about 500 colonies were separated into 12 pools (A1-A12) and plasmid DNA from these pools was isolated using standard methods and dot blotted again for the purpose of secondary screening. The membrane was probed with labeled AtNHX1 1 fragment as done previously for the primary screening. The A12 from Pc27 pool and A3 from p37 pool showed intense signals. Plasmid DNA from A12 and A3 (p37) were transformed to E. coli DH5. The colonies were separated into 11 pools again (B1-B11) as described above. Dot blot analysis showed that B1 from Pc27-A12 and B4 from P37-A3 showed highest signal. Plasmid DNA was isolated and used for E. coli transformation. Hundred individual colonies were patched from each plate and separated into 12 pools designated as or termed as (C1-C12).C3 from P37 check showed intense signal. Plasmid DNA was isolated from 12 individual colonies (corresponding to C3 pool) and one of the colonies hybridized intensely with the AtNHX1 probe. Plasmid DNA from this colony was isolated and purified by PEG precipitation method. PCR screening was carried out using M13 F and Rev universal primers generated a 2 kb fragment. This clone was further sub-cloned into PBSKII vector to get a complete sequence. The nucleic acid sequence of both strands of the full length cDNA was determined using the dideoxy chain termination method (Sanger et al., 1977) with an ABI 310 automated DNA sequencer (Perkin-Elmer) and M13/pUC18 forward and reverse primers. Protein translation, nucleic acid and amino acid sequence alignments were performed using BLASTX and BLASTN options at the NCBI website (Altschul et al. 1997).

### Example 5

### Random Amplification of cDNA Ends

In order to procure the missing sequence information at the 5′ end of PcNHX1, 5′ RACE (Rapid Amplification of cDNA Ends) was performed. Primers were designed based on the sequence information from the first clone. Total RNA from 0.5M NaCl, 48 hr stressed Porteresia coarctata leaves was used to isolate total RNA. The mRNA was enriched from the total RNA population using streptavidin paramagnetic particles (Sigma, S2415) and biotin labeled oligo d (T) 18 primer. The enrichment process is as follows: 250 (g total RNA in 10 mM TrisCl, pH 7.5; 0.5M KCl (Buffer A) was heated to 65(C with 200 ng biotin labeled oligo d (T) primer and chilled on ice. Biotin-captured mRNA was immobilized by incubation with streptavidin paramagnetic bead suspension (equilibrated in Buffer A). The beads were washed with 10 mM Tris Cl, pH 7.5; 0.25M KCl and the mRNA eluted in DEPC water and concentrated by lyophilization. The concentration and integrity of the eluted mRNA was checked on a 1.2% formaldehyde-agarose gel (Sambrook et al. 1989).

First strand cDNA was synthesized using the SMART™ RACE kit (Clontech) according to the manufacture's instructions. When the 5′ RACE reaction following first strand synthesis was run on a 1.5% agarose gel, multiple bands were observed. Southern analysis revealed a band corresponding to 450 bp, which gave the highest signal. This PCR fragment was gel purified and cloned in T/A cloning vector. The 450 bp fragment identified by 5′ RACE and the original 1.875 kb fragment were joined by Splice Overlap Extension (SOE) PCR. The resulting 2.148 bp fragment was gel purified and cloned in T/A cloning vector. Universal primers were used for generating the compiled sequence of the clone.

### Example 6

### Tail PCR for Isolation of PcNHX1 Promoter Sequences

For TAIL-PCR, the gene specific primers originally designed for 5′-RACE were used in combination with arbitrary degenerate primers, originally described by, Leu et al. For primary reaction, Porteresia coarctata genomic DNA was used as template. Two successive rounds of PCR reactions were carried out using the products of previous PCR as templates employing a common arbitrary primer in combination with a gene specific primer in a consecutive manner. The products of primary, secondary and tertiary reactions were separated on adjacent lanes in a 1.5% agarose gel, and discreet PCR products, showing difference in size corresponding to the relative positions of the gene specific primers were identified. The results were confirmed through Southern blot analysis using the 5′RACE product as a probe.

### Example 7

### Primer Extension Analysis of the Promoter

In order to accurately map the transcription start site, primer extension assay was carried out using an antisense oligonucleotide derived from the 5′UTR region immediately upstream of the start codon of the PcNHX1 gene.

### Example 8

### Northern Analysis for Studying Induction Kinetics of PcNHX1

To study the effect of salinity ABA on the expression of the PcNHX1, Northern analysis was performed using total RNAs, isolated from stressed Porteresia coarctata leaves (for growth conditions and RNA isolation, see EXAMPLE 1).

For Northern analysis, equal amounts of total RNA 30 (μg) were electrophoresed on a 1.2% MOPS-formaldehyde gel, transferred to nylon membrane (Hybond N, Amersham) and fixed by UV cross linking according to the manufacturers instructions. A PCR amplified fragment of the PcNHX1 clone was used as a probe.

### Example 9

### Construction Plant Transformation Vectors

Yet another embodiment of the present invention relates to cloning the PcNHX1 insert in the sense direction into the BamH1 site of the binary vector pCAMBIA 1301 downstream of CaMV 35S promoter. The gene was also cloned in the antisense orientation into the same restriction site in the same vector. A third construct was made by cloning the PcNHX1 promoter sequence upstream of the PcNHX gene in pCAMBIA 1301. The PcNHX promoter was cloned in the Sal1 and BamH1 sites of the promoter fusion vector pCAMBIA 1391Z upstream of a promoter less GUS gene.

### Example 10

### Plant Transformation

### Transformation of Tobacco Using Agrobacterium

Plant transformation vector constructs PcNHX-1301, PcNHX AS-1301, PcNHX-PRM-1301, and PcPRM-1391z were transferred to tobacco explants. Transformation of tobacco leaf discs was performed through Agrobacterium co-cultivation. The Agrobacterium strains used were LBA4404 for PcPRM-1391z and EHA105 for PcNHX-1301, PcNHX AS-1301, and PcNHX-PRM-1301. These constructs were mobilized into Agrobacterium tumefaciens strain EHA105 by the freeze-thaw method. Agrobacterium-mediated transformation of tobacco (Nicotiana tabacum) cv. Wisconsin was carried out by the standard protocol. Briefly, sterile tobacco leaf discs were cut and transferred to Murashige and Skoog (MS) medium containing 3% sucrose, 1 mg/L BAP, 1 mg/L NAA, 0.8% Bacto-Agar, pH 5.6 at 28° C. in 16 hours light and 8 hours darkness for 24 hours prior to transformation. 100 ml of an overnight grown culture of Agrobacterium strain containing the construct was resuspended in 0.5× MS liquid medium with 3% sucrose, pH 5.6 (5 ml). The leaf discs were subsequently co-cultivated with the resuspended Agrobacterium for 30 minutes. The discs were dried on sterile Whatmann No. 1 discs and transferred to MS medium containing 3% sucrose, 1 mg/L BAP, 1 mg/L NAA, 0.8% Bacto-Agar, and pH 5.6 at 28° C. in 16 hours light and 8 hours darkness for 48 hrs. The leaf discs were given several washes in liquid MS medium with 3% sucrose, pH 5.6 containing 250-mg/mL cefotaxime. Excess moisture on the leaf discs was blotted on sterile Whatmann No. 1 filter paper. The discs were then placed on selection media, that is, MS medium containing 3% sucrose, 1 mg/L BAP, 1 mg/L NAA, 0.8% Bacto-Agar, pH 5.6 containing 250 mg/mL cefotaxime and 25 mg/L hygromycin at 28° C. in 16 hours light and 8 hours darkness. The leaf discs were transferred to fresh selection media every 14 days until multiple shoot regeneration was seen. Shoot regeneration was seen between 20-35 days after first placing on the selection media. Independent shoots were then transferred to rooting medium (MS medium containing 3% sucrose, 0.8% Bacto-Agar, pH 5.6 containing 250 mg/mL cefotaxime and 25 mg/L hygromycin at 28° C. in 16 hours light and 8 hours darkness). After establishment of roots in the medium the plants transferred to fresh rooting medium every 14 days, each time transferring a shoot cut from the previous plant. Transformation of plants was confirmed by glucouronidase (GUS) staining of stem, leaf and root sections of the plant. The protocol for GUS staining was according to Jefferson R A et al., 1987. The rooted plants were further transplanted to soil and maintained in the greenhouse.

### Example 11

### Transformation of Rice Using Agrobacterium

Two cultivated varieties of indica rice, Pusa Basmati and IR-20, were used for transformation using Agrobacterium method. These were used for transformation. Transformation of rice calli was performed by standard Agrobacterium method which is well known to the people skilled in art. The constructs used for transformation, PcNHX-1301 and PcNHX-PRM-1301 were mobilized into the Agrobacterium super virulent strain EHA105 by freeze thaw method. The protocol followed for both Pusa Basmati and IR-20 were similar (see EXAMPLE) except for the fact that the callus induction medium used was MS+3 mg/L 2,4-D.

### Transformation Method

The embryos were dehusked and surface sterilized with 70% EtOH for 1 minute followed by sterilization with 2% sodium hypochloride for 2 hours. The seeds were subsequently washed in sterile distilled water (5-6 times) and dried on a sterile blotting paper. Finally, the seeds were plated on MS medium containing 2 mg/L 2,4-D for callus induction for 3 weeks. Embryogenic calli were cut into small pieces and pre-cultured for two days before Agrobacterium infection.

### Agrobacterium Infection

A single colony of the Agrobacterium harbouring the construct was inoculated into 5 ml YEP containing 10 mg/L Rifampicin and 50 mg/L Kanamycin. Twenty five microlitres of this culture was inoculated into 50 ml of YEP containing the antibiotics. The cells were harvested when the culture reached an O.D of 0.8 by centrifuging culture at 5000 rpm for 10 minutes (room temperature). The pellet was re-suspended in 5 ml 3% liquid MS and pelleted down again and was finally re-suspended in 5 ml 3% MS medium. Two days pre-cultured calli was transferred to a petri dish containing 5 ml 3% MS. The bacterial suspension was added to this and the plate was swirled gently for 2 minutes. After two minutes, the calli was removed from the plate and dried on sterile filter paper. The calli were transferred to the same medium used for callus induction and left for co-cultivation for 48 hrs.

### Washing and First Selection

After 48 hrs the calli were washed in 3% MS containing 250 mg/L cefotaxime. The washing was repeated 7-8 times. After washing the calli were dried on filter paper and transferred to selection media. Selection was carried out on plates containing MS+50 mg/L hygromycin+250 mg/L cefotaxime.

Second selection After 15 days the growing calli were transferred to fresh selection plates.
Third selection After two weeks the calli was transferred again to fresh selection media and cultured for 2 more weeks.
Regeneration After 3 rounds of selection, embryogenic calli were transferred to regeneration media i.e., MS+1.5 mg/L BAP+0.5 mg/L Kinetin+0.5 mg/L NAA without antibiotics. The shooted calli was transferred to 3% MS without hormones and with antibiotic for rooting.

### Example 12

### Transformation of Rice Through Particle Bombardment

For particle bombardment, plasmid DNA was coated on to gold particles and delivered into the calli by using a Bio-Rad PDS-1000/He Biolistic System. Following steps were undertaken:

Calli developed from mature seeds were excised into small pieces and transferred to MS plates containing 3 mg/L 2,4-D and sorbitol and Mannitol, 0.2M each. Simultaneously, micro-carriers were prepared. 1.5 mg of gold particles (Biorad, USA) was weighed on a microfuge tube. 10 μg of plasmid DNA was taken in 100 μL Xho Buffer (30 μL of 5M NaCl, 5 μL of 2M TrisCl, pH 8, 965 μL distilled water) and added to the tube containing the gold particles. The contents of the tube were mixed by vortexing. Spermdine (0.1M, 100 μL) was added to the tube and mixed by vortexing. Hundred microlitres of 2.5M CaCl2 was added drop-by-drop while vortexing and incubated on vortex for 10 minutes. The mixture was centrifuged briefly at 13,000 rpm and the supernatant was discarded. The gold pellet was re-suspended in 1 mL 100% EtOH, pelleted and re-suspended in 1 mL 100% EtOH. The contents were vortexed in the suspension briefly and stored in −20° C. For each bombardment, 15 μL of this gold suspension was used after sonicating briefly. After 4 hrs, the macro-carrier launch assembly was sterilized using 70% EtOH. The micro-carrier suspension was vortexed and 15 μL of the micro-carrier suspension was coated on the macro-carrier. Finally, the macro-carrier was placed inside the holder with a sterile forceps. The disc was ruptured and the ruptured disc was inserted to the helium accelerator tube. The stop screen was inserted into the macro-carrier launch assembly. The macro-carrier holder was inserted inside the assembly, and placed inside the chamber. The petri plate containing the calli was placed on the dish holder and inserted into the chamber. The vacuum pump was switched on and was kept at hold. The helium pressure was allowed to build up by pressing the fire switch on. After the bombardment, the vacuum was released by pressing the vent switch. The used rupture disc was removed and macro-carrier was replaced with new ones. Once all the bombardments were over, the plates were returned to 28° C. and incubated for 4 hours. After 4 hours the bombardment was repeated again, and the plate was incubated overnight on the same osmoticum plates. After 16 hrs, the call was transferred to the selection media. After three rounds of selection, the calli was transferred to regeneration media without any antibiotics. The regenerated plants were transferred to MS media with antibiotics and no hormones. The plants were finally transferred to Yoshida solution for hardening in the green house. After 3 weeks, the plants were transplanted to soil.

### Example 13

### Salt Stress Analysis in Transgenic Tobacco and Rice Plants

### Whole-Plant Salt Stress Treatments:

The salt tolerance conferred by over-expressing PcNHX1 in tobacco and rice transgenics was analyzed by performing whole plant salt stress treatments. Phenotypic growth retardation study was also performed between control and transgenic plants. Three control and transgenic plants were grown initially in ½ MS for 1 week. Later, they were transferred to ½ MS medium supplemented with 150 mM and 200 mM NaCl. It was observed that in 150 mM NaCl, transgenic plants showed better rooting when compared to control plants. At 200 mM NaCl, both control and transgenic plants did not root. It was also found that the both tobacco and rice transgenic plants suffered less damage in 150 mM and 200 mM NaCl stress. Phenotypic growth retardation was not evident in transgenic plants (tobacco and rice) grown in pots and irrigated with 150 mM NaCl solution for 1½ weeks.

### Example 14

### GUS Assay for NHX Promoter Activity in Tobacco

The functionality of PcNHX1 promoter in transgenic plants was tested by fusing the GUS coding sequence to PcNHX1 promoter (see EXAMPLE for construction of these reporters constructs). The constructs thus generated were mobilized into tobacco plants. Leaves from the transgenic tobacco plants carrying the promoter-GUS fusion plasmid, were subjected to a preliminary GUS analysis. Pieces of fresh leaves were incubated in substrate solution (X-Gluc in NaPO4 buffer) overnight.

1-40. (canceled)

41. An isolated cDNA sequence from Porteresia coarctata comprising a polynucleotide sequence as set forth in SEQ ID NO: 1, wherein expression of said polynucleotide sequence in a plant results in conferring tolerance to salt stress as compared to a corresponding wild type plant.

42. The cDNA as claimed in claim 41, wherein said cDNA encodes a polypeptide having amino acid sequence as set forth in SEQ ID NO: 2.

43. A recombinant vector comprising of a regulatory sequence operably linked to the cDNA as claimed in claim 41.

44. The recombinant vector as claimed in claim 43, wherein the regulatory sequence is selected from a group consisting of CaMV 35S promoter, actin promoter, maize ubiquitin promoter and alcohol dehydrogenase promoter.

45. The recombinant vector as claimed in claim 43, wherein the recombinant vector is a recombinant plant transformation vector.

46. The recombinant vector as claimed in claim 45, wherein the recombinant plant transformation vector is selected from a group consisting of PcNHX-1301, PcNHX AS-1301, PcNHX-PRM-1301, and PcPRM-1391z.

47. A host cell comprising the recombinant vector as claimed in claim 43 to produce a recombinant host cell.

48. The host cell as claimed in claim 47 is selected from a group consisting of E. coli, Agrobacterium and yeast.

49. The host cell as claimed in claim 48, wherein the E. coli is selected from a group consisting of as JM101, DH5α, BL21, HB101, and XL1-Blue.

50. The host cell as claimed in claim 48, wherein the Agrobacterium strain is selected from a group consisting of LBA4404, EHA101, EHA 105, GV3101 and A281.

51. A method for conferring salt stress tolerance in plant, said method comprising transformation of said plant with recombinant vector as claimed in claim 43 to produce transformed plant cells, culturing the transformed plant cells to obtain a salt stress tolerant plant.

52. The method as claimed in claim 51, wherein the transformation is performed by a method selected from a group consisting of Agrobacterium mediated transformation, particle bombardment, vacuum-infiltration and in planta transformation.

53. The method as claimed in claim 51, wherein said transformation is performed by Agrobacterium mediated transformation.

54. The method as claimed in claim 53, wherein the Agrobacterium mediated transformation comprises of:
a. obtaining a suitable explant from a plant,
b. co-cultivating the explant with an Agrobacterium strain that comprises of a recombinant vector as claimed in claim 43 to produce transformed plant cells,
c. culturing the transformed plant cells to produce the salt stress tolerant plant.

55. The method as claimed in claim 51, wherein the plant is selected from a group consisting of dicot and monocot.

56. The method as claimed in claim 55, wherein the dicot plant is selected from a group consisting of tobacco, tomato, pea, soybean, Brassica, chickpea and pigeon pea.

57. The method as claimed in claim 55, wherein the monocot plant is selected from a group consisting of rice, maize, wheat, barley and sorghum.

58. The method as claimed in 54, wherein the explant is selected from a group consisting of cotyledons, hypocotyls, leaves, stem and roots.

59. A transgenic plant having tolerance to salt stress comprising the polynucleotide sequence as set forth in SEQ ID NO: 1.

60. The transgenic plant as claimed in claim 59, wherein said plant is selected from a group consisting of dicot and monocot.

61. The transgenic plant as claimed in claim 60, wherein the dicot plant is selected from a group consisting of tobacco, tomato, pea, soybean, Brassica, chickpea and pigeon pea.

62. The transgenic plant as claimed in claim 60, wherein the monocot plant is selected from a group consisting of rice, maize, wheat, barley and sorghum.

63. A progeny of said transgenic plant as claimed in claim 62.

64. An isolated promoter functional in plant cells comprising a polynucleotide sequence as set forth in SEQ ID NO: 3.

65. An isolated promoter functional in plant cells comprising at least 200 contiguous nucleotides of the polynucleotide sequence as set forth in SEQ ID NO: 3.

66. A recombinant vector comprising the promoter of claim 64 or 65 operably linked to a heterologous DNA sequence of interest.

67. The recombinant vector as claimed in claim 66, wherein the heterologous DNA sequence encodes a protein selected from a group consisting of insect resistance protein, a bacterial disease resistance protein, a fungal disease resistance protein, a viral disease resistance protein, a nematode disease resistance protein, a herbicide resistance protein, a protein affecting grain composition or quality, a selectable marker protein, a screenable marker protein, a protein affecting plant agronomic characteristics and a stress resistance protein.

68. A transgenic plant comprising a promoter as claimed in claim 64 or 65.

69. The transgenic plant as claimed in claim 68, wherein the plant is selected from a group consisting of monocot and dicot.

70. A progeny of said transgenic plant as claimed in claim 68.

71. A method of producing a transgenic plant comprising transformation of a plant with recombinant vector of claim 66 to produce transformed plant cells, culturing the transformed plant cells to produce a transgenic plant.

72. The method as claimed in claim 71, wherein the transformation is performed by a method selected from a group consisting of Agrobacterium mediated transformation, particle bombardment, vacuum-infiltration and in planta transformation.

73. The method as claimed in claim 72, wherein said transformation is performed by Agrobacterium mediated transformation.

74. A method as claimed in claim 73, wherein the Agrobacterium mediated transformation method comprises of:
d. obtaining a suitable explant from said plant,
e. co-cultivating the explant with an Agrobacterium strain that comprises of a recombinant vector as claimed in claim 66 to produce transformed plant cells,
f. culturing the transformed plant cells to produce salt stress tolerant plant.

75. The method as claimed in claim 71, wherein the plant is selected from a group consisting of dicot and monocot.

76. The method as claimed in claim 75, wherein the dicot plant is selected from a group consisting of tobacco, tomato, pea, soybean, Brassica, chickpea and pigeon pea.

77. The method as claimed in claim 75, wherein the monocot plant is selected from a group consisting of rice, maize, wheat, barley and sorghum.

78. The method as claimed in claim 54, wherein the plant is selected from a group consisting of dicot and monocot.

79. The method as claimed in claim 74, wherein the plant is selected from a group consisting of dicot and monocot.


SEQUENCE LISTING (4 sequences)

SEQ ID NO: 1 - DNA, 2270 bp, Porteresia coarctata
gattttgttcatatcgaattccagatttccaatccatccacccaatttctcgagcaagagcaatcccgagttcttttcttggaattttttttctgccccttctcgaattaggcattccccaacgaacaagagctcgggggcggtggattggttggttaaagctcgcatcttgccgcggggaatctctcttctcttctctaaggtggtggccgaagcagtcgccgccggtgaggcatggggctggaggtggcggcgcgcctggtggcgcggctaggggcgctgaacacgacctccgactacgcgtcggtggtgtccatcaacctgttcgtggcgctgctctgcggctgcatcgtcctcggccacctcctcgaggagaatcgatgggtcaacgagtccatcaccgcgctcatcatcgggctctgcaccggcgtggtgatcttgctgacgaccaaagggaagagctcgcacattttcgtcttcagcgaggatctgttcttcatctacctcctccctccgatcatcttcaacgcgggttttcaggtaaagaaaaagcagttcttccgaaatttcatgacgatcacattatttggagctgttgggacaatgatatcctttttcacaatagctattgctgccattgcaatattcagcagaatgaacatcggaacgctggatgtaggagattttcttgcaattggagccatcttttctgcaacagattctgtgtgcacattgcaggtcctcaaccaggatgagacgccctttttgtatagtctggtattcggtgaaggtgttgtgaacgatgccacatcaattgtgcttttcaacgcactacagaactttgatcttgtccacatagatgtggctgtcgtcctgaaattcttggggaacttcttttatttatttttgtcaagcaccttccttggagtatttgctggattgctcagtgcattcataatcaagaagttatacattggaaggcattctactgaccgtgaggttgcacttatgatgctcatggcttacctctcgtatatgctggccgagttgctcgatttgagtggtattctcaccgtgttcttctgtggtattgtgatgtcacattacacttggcataacgtcacagagagttcaagagttacaaccaagcacgcatttgcaactttgtccttcattgctgagacttttctcttcctgtatgttgggatggatgcattggatattgaaaaatgggagtttgccagtgacagccctggcaaatccattgggataagctcaattttgctaggattggttctggttggaagagctgcttttgttttccctttgtcgttcttgtcgaacttaacaaagaaggcaccatatgaaaaaataacctggagacagcagattgtaatttggtgggctgggctgatgagaggagctgtgtcgattgcccttgcttacaataagtttacaagatctggccatactcagctgcacggcaatgcaataatgatcaccagcaccatcactgtcgttctttttagcactatggtgtttgggatgatgacaaaacctttgatacggctgctgctaccggcctcaagccacaccgtcacctctgagccctcatctccaaagtccttgaattctcctctcctgacaagcatgcaaggttctgacctcgagagttcaactcacattgtgaggccttccagcctccggatgctcctcagcaagccgacccacactgtccactactactggcgcaagttcgatgatgcactgatgcgacccatgttcggtgggcgtgggttcgtgcccttctcccctggatcacccaccgaacagagcgtccatgaaggacaatgaacaatgcaaagaaataagaatgattgatgaagaggagacatgtaaaacgtgacagcaaaagagaaggcgagttttgggtttgtagaatttggctgctgctaatgagttgttgatagtgcttatattcttcagaacttcagatggtacctcaccaaggcctaagaggaccttctgataagggttcagatggagtgatagatgattggtttcatctatcaggatgaaccctagtgagggacatagggtgatgtgctccgacaacctgtaaattttgtagattagcagccatttgtacctatctaccatctttagctagcaggtcttttcctatttgccaccctttatgtaaaatgaggttctcccaaatagatttgtgtgtaaaatgattttgcttggttgataaaaaaaaaaaaaaa

SEQ ID NO: 2 - protein, 539 aa, Porteresia coarctata
MGLEVAARLVARLGALNTTSDYASVVSINLFVALLCGCIVLGHLLEENRWVNESITALIIGLCTGVVILLTTKGKSSHIFVFSEDLFFIYLLPPIIFNAGFQVKKKQFFRNFMTITLFGAVGTMISFFTIAIAAIAIFSRMNIGTLDVGDFLAIGAIFSATDSVCTLQVLNQDETPFLYSLVFGEGVVNDATSIVLFNALQNFDLVHIDVAVVLKFLGNFFYLFLSSTFLGVFAGLLSAFIIKKLYIGRHSTDREVALMMLMAYLSYMLAELLDLSGILTVFFCGIVMSHYTWHNVTESSRVTTKHAFATLSFIAETFLFLYVGMDALDIEKWEFASDSPGKSIGISSILLGLVLVGRAAFVFPLSFLSNLTKKAPYEKITWRQQIVIWWAGLMRGAVSIALAYNKFTRSGHTQLHGNAIMITSTITVVLFSTMVFGMMTKPLIRLLLPASSHTVTSEPSSPKSLNSPLLTSMQGSDLESSTHIVRPSSLRMLLSKPTHTVHYYWRKFDDALMRPMFGGRGFVPFSPGSPTEQSVHEGQ

SEQ ID NO: 3 - DNA, 1773 bp, Porteresia coarctata
tgtcgagtgaaatgaaaagaaaagaaaagagaaaatgtagttcttgcttgaaacgtaatcaaggaactttcttgtgagttttcgatggctgcaggctctttctgaatccctatcaccattcattgaatcgtcgataaatcacgagcaaagtgcagcacgggaagatcgatctgcagaaaaaaaaaacaccagaactgaaaggacgatgaaatgcataaatcaatggaagaatgcatagtggataatgcatctctctcctgaattaaatttgactcctacagcgttcaagggccagattaataaggatctaggaaaaaatgattgatctcaaatctgcttaaataatgtgctctactggtgtaaacacatggagttgtctccattatatttcaagaaaacggttattttctgtaagtatacataagcaataatctttaaaaaaatcacaggagaaaaacaaaacatgtcaagcataaacagcagagagtcgtatactaataaatttacagctgtatttcaaatatagcctgcactgttcagtgttaaattagaaaaaaaaaaggagcacatgatgaattatcacaaaccgaaacagaaaatggccgcatttcacatgtgcaaaaagaagaataaaaccagaaaattaagagcatgacagcaagacacagtcctgcttgaaacattttttccaaagaaaattcaaatttaaacaggaaagagtggcaaacatacccccattccttactacaaatacgggtggatgcagcggatccagctggacaatccaaattggcagggttgtcgcctgtcggggcagatcacgctccgcgcggccccacccgctgtgtcccaccaaacaccacgcaccaaacacaatccattaaaataccgccacgtgccacccctgcgggacccagcccctccgctttatatacggctcatcacgagtgcatccaaaaaattacgaaaaagtttggggcgctcgcgcgaaggcgaaaccaaccgagaggtctggacgaaagaacaaccaaattccaatttcccaacccgcaattcagaatccccccgttcctcacctgaatcagcgttttgcgcggaagatttcatctgttcatcttcggatctttgttttctctgattttaccggagttttctcggcctggaaaggataataagtttggattttgttcatatcgaattccagatttccaatccatccacccaatttctcgagcaagagcaatcccgagttcttttcttggaattttttttctgccccttctcgaattaggcattccccaacgaacaagagctcgggggcggtggattggttggttaaagctcgcatcttgccgcggggaatctctcttctcttctctaaggtggtggccgaagcagtcgccgccggtgaggcatggggctggaggtggcggcgcgcctgtggcgcggctaggggcgctgaacacgacctccgactacgcgtcggtggtgtccatcaacctgttcgtggcgctgctctgcggctgcatcgtcctcggccacctcctcgaggagaatcgatgggtcaacgagtccatcaccgcgctcatcatcgtatgcatagaccgaccgaatggctcgcctttgttgggtttccttctccatggattgattggttgctgccaaggcgaccgcctttgctaacgctcgtttgctgcttgatgatatgcttgcttggccaggggctctgcaccggcgtggtgatcttgctgacgaccaaagggaagagctcgc

SEQ ID NO: 4 - DNA, 29 bp, Artificial Sequence
  Synthetic
gactagttctagatcgcgagcggccgccc